SEQUENCE LISTING (98 sequences)

SEQ ID NO: 1 - protein, 98 aa, Homo sapiens
WGCSKEVWLNMLKKESRYVHDKHFEVLHSDLEPQMRSILLDWLLEVCYSNTNFLCLCMMETYLAVNVLSMYSQEFIVHPQEAVFFEVLTVVDCTQSGK

SEQ ID NO: 2 - protein, 210 aa, Homo sapiens
DCEYCQYHVQAQYKKLSAKRADLQSTFSGGRIPKKFARRGTSLKERLCQDGFYYGGVSSASYAASIAAAVAPKKKIQTTLSNLVVKGTNLIIQETRQKLGIPQKSLSCSEEFKELMDLPTCGARNLKQHLAKATASGTISCMATRVSLCSLSLPGHPLSLGFCFRDYGEPKTSHQVHLGLSTLEATEAADVGDEEKEIRRNTEAISAELK

SEQ ID NO: 3 - protein, 45 aa, Homo sapiens
ICVTCKEVKQPQELGRGGLGAGGGRHHLGRRHLSALTHSIPSVKT

SEQ ID NO: 4 - protein, 8 aa, Homo sapiens
TRLNRRSA

SEQ ID NO: 5 - protein, 9 aa, Homo sapiens
FRHQMALGD

SEQ ID NO: 6 - protein, 120 aa, Homo sapiens
MVNSNRKWEARRGPLCVSFSVSPTLVDLFTSSVTPCLSLPHSAFSHIWQLLPTSAACSLSASLFLPLAPHPLSFMSVVYLSLLSSLCNCPIPLHCSFIFSQPAIQHTFTEHLLCWENIHE

SEQ ID NO: 7 - protein, 133 aa, Homo sapiens
NHTVAFLGTSDGRILKVWPRLGRGGSGGPVCAQGPYPHRPALAHTGVPHPRWHLLRVRLYPCGDKQESQARPGTVWRPGQPVRHDPGQGEPDAAPESTLGQVCPVIASQALRTGLGARGGWGAAELGPSPWSG

SEQ ID NO: 8 - protein, 31 aa, Homo sapiens
RGCRLTELGLMHDHKPAAGPGVSAGWATGAL

SEQ ID NO: 9 - protein, 28 aa, Homo sapiens
LEFLTATGVQETFVFCCWKAAQIKEHLL

SEQ ID NO: 10 - protein, 87 aa, Homo sapiens
GGAAQRSGERRARGRREVEPFGGRSPVKDGLFPPAAAAGGGALKRACLGIFSLSLEPVLWTLRSRVLLYHLTFFSFQGQGPHLLLSL

SEQ ID NO: 11 - protein, 8 aa, Homo sapiens
ICAYCHTR

SEQ ID NO: 12 - protein, 35 aa, Homo sapiens
KQQIESEVSSQGSRQTQTPPAWATCPLQREFGQWL

SEQ ID NO: 13 - protein, 36 aa, Homo sapiens
PYMTPVQVSVPWRGGVRLGEGQAPAASDPQEVIGQI

SEQ ID NO: 14 - protein, 8 aa, Homo sapiens
ASYPSDHW

SEQ ID NO: 15 - protein, 27 aa, Homo sapiens
LFVPNLIGECCPRPRAEREGEGPWRAP

SEQ ID NO: 16 - protein, 65 aa, Homo sapiens
NLPTSLEGLSNLAGQAAPGAPLSSGPLWALLLPWVEGVEPCSSDQYPEGKGWSYVTVTFAIMMMP

SEQ ID NO: 17 - protein, 40 aa, Homo sapiens
LQGERISGNLDAPEGGFDAILQTAVCTVGTGKGAASLGQG

SEQ ID NO: 18 - protein, 63 aa, Homo sapiens
SEKHLISGEPLWGAPVLGETAQMGVWRQEDENFHWGGTLCRKRALAERIDLGWDCNCRSERIP

SEQ ID NO: 19 - protein, 170 aa, Homo sapiens
VRGLVLPGEAWAPCAGMGARRVSPAPSDLDFGFLSFLCILNPFSLGLAIPPALSWGWLSPRPPPVKAASPPASRRTLRCHSLSSPPAASAPRPPAVPGMDISGKGHASGDLLEPWPLGRNLVACWSLWDDGSPMGHWEGGTLGAMEAHVLCDGCCELSYPSWCLMGPWVV

SEQ ID NO: 20 - protein, 398 aa, Homo sapiens
QSFLLLLLLPACWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAASFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLRVAHSAPVGAQLATELSIAEGQEVLFGVFVTGKDGGPGVGPNSVVCAFPIDLLDTLIDEGVERCCESPVHPGLRRGLDFFQSPSFCPNPVS

SEQ ID NO: 21 - protein, 8 aa, Homo sapiens
LLAAYFFR

SEQ ID NO: 22 - protein, 8 aa, Homo sapiens
EIRGKPIK

SEQ ID NO: 23 - protein, 76 aa, Homo sapiens
DVLEFNQVILPSACPGAQDLREVTGPQGLCGPDCHSFPQVIIFVKSVQRCMALAQLLVEQNFPAIAIHRGMAQEER

SEQ ID NO: 24 - protein, 63 aa, Homo sapiens
RRARAKAGEGLPGAGAACWGAGQQQGPTSPLPLCCARSLCREEGQQCCCPAHRELPGQSDLPR

SEQ ID NO: 25 - protein, 39 aa, Homo sapiens
DEEAMEKVSVLFLGSREGEGQKPGSEEQAQKIWGGGTLL

SEQ ID NO: 26 - protein, 55 aa, Homo sapiens
LQELHAKVESQSPLPTWATPLSLWGWGSSLHFWKEYSNHWPVFSGAVTGILTSVA

SEQ ID NO: 27 - protein, 19 aa, Homo sapiens
LIQGLKEVRVLGSRLVIVE

SEQ ID NO: 28 - protein, 30 aa, Homo sapiens
KPTFIKGVSSSPCQVVLKKCLLAGCSGSHL

SEQ ID NO: 29 - protein, 24 aa, Homo sapiens
FSPAPKLGESVRVQESTPHKGKDW

SEQ ID NO: 30 - protein, 15 aa, Homo sapiens
ELRQRKRGREPVALP

SEQ ID NO: 31 - protein, 54 aa, Homo sapiens
PEDYGEEVKATLSFPQSCSFWSRLFEVSCAETRDGWAPDGALEALGSCSLRPCP

SEQ ID NO: 32 - protein, 15 aa, Homo sapiens
VIFILMKVGSSRVHC

SEQ ID NO: 33 - protein, 35 aa, Homo sapiens
DEGLEETGMTPLSGVLKGILRGCHCWTGEGSVGDQ

SEQ ID NO: 34 - protein, 33 aa, Homo sapiens
VSADSMQVWQCGWPGRLGTPEAAWTPCGKGLKG

SEQ ID NO: 35 - protein, 25 aa, Homo sapiens
TTLDDPLGKGPILFPWDRWNRTLWC

SEQ ID NO: 36 - protein, 29 aa, Homo sapiens
QLRKNQQVRQAVLLSPRWLSHSQVLAPRL

SEQ ID NO: 37 - protein, 83 aa, Homo sapiens
SHLSLAQGESDTPGVGLVGDPGPSRAMPSGLSPGALDSDPVGLGDPLSEISKLLEAGKEGWAREVWEGNGDAWRDECQDFGGL

SEQ ID NO: 38 - protein, 85 aa, Homo sapiens
SPVSKLQVKLEPGTCSSGPAPTTGDVTPQGLLACPSCVLAVASLSCTVPGPLPRPSWEPLARARPPQAPTATAASLALSSSHSGV

SEQ ID NO: 39 - protein, 16 aa, Homo sapiens
HMVGDKPGGPHPRPGS

SEQ ID NO: 40 - protein, 8 aa, Homo sapiens
AVPAGAKM

SEQ ID NO: 41 - protein, 43 aa, Homo sapiens
VLPSFYQVRRFLCAVSWQERESSPHACPPIPLGVLLRGLRWHF

SEQ ID NO: 42 - protein, 9 aa, Homo sapiens
GILAALQGA

SEQ ID NO: 43 - protein, 37 aa, Homo sapiens
QELRALQGRLRAQGLGPALLHRPLFAFPDAVRAPSGS

SEQ ID NO: 44 - protein, 76 aa, Homo sapiens
PAIAFGGRWAPPPASAPPPSPAPTQRPQPFSPLPSDSHRCLFVRPQAVVPGLVLPPPGSPPTCPPSHLLSLDVRPH

SEQ ID NO: 45 - protein, 14 aa, Homo sapiens
AAGGSSEGKGMAGG

SEQ ID NO: 46 - protein, 32 aa, Homo sapiens
DMAIAMAVSYTPAWGLQGTQELTMVGCAEGQG

SEQ ID NO: 47 - protein, 63 aa, Homo sapiens
PRNYLNTLSTALNILEKYGRNLLSPQRPRYWRGVKFNNPVFRSTVDAVQVNPLALWERGLGFG

SEQ ID NO: 48 - protein, 21 aa, Homo sapiens
HQRFQFSRWVPIWRRPAVSLK

SEQ ID NO: 49 - protein, 20 aa, Homo sapiens
QTLGSLKACLPVLTPSIHQV

SEQ ID NO: 50 - protein, 108 aa, Homo sapiens
AQVPQASGEQPRGNGANPPGAPPEVEPSSGNPSPQQAASVLLPRCRLNPDSSWAPKRVATASPFSGLQKAQSVHSLVPQGEKPDGFSARVQGVWNTCTCWRREGLVWP

SEQ ID NO: 51 - protein, 35 aa, Homo sapiens
FSQLGFIGVPLPLPDGAFQSHLPLPSSLPVPLCPL

SEQ ID NO: 52 - protein, 75 aa, Homo sapiens
PTIGYCQVRGLTGAQRQHLPPEVHRSRSLWSQACVVTLPCVPRKEPSRKRCNLKTVLIAHRIPMPNRSRRLCTFV

SEQ ID NO: 53 - protein, 31 aa, Homo sapiens
MGHRYVEGLIYIALVTVNKALNNRGLTPSEF

SEQ ID NO: 54 - protein, 16 aa, Homo sapiens
EVCRVCQVSAAPGSTA

SEQ ID NO: 55 - protein, 29 aa, Homo sapiens
PEVLQKGVAYDSSADWFSLGCMLFKLLRG

SEQ ID NO: 56 - protein, 28 aa, Homo sapiens
TGSGFRVGEGRGHGWGQGKDRHQARWES

SEQ ID NO: 57 - protein, 33 aa, Homo sapiens
CCKTLDQVSVVLSTSNTKVVKHSIRSVTEAKVR

SEQ ID NO: 58 - protein, 45 aa, Homo sapiens
FEEPELRVGPRSRPRPGPAQPPSLPLQLGSVAPSVSLGVSYAGPG

SEQ ID NO: 59 - protein, 8 aa, Homo sapiens
LWEELGHR

SEQ ID NO: 60 - protein, 14 aa, Homo sapiens
AVMRILSGERGCQG

SEQ ID NO: 61 - protein, 27 aa, Homo sapiens
ETDFGGDVSLGTPGPEGGGAGGLDSWV

SEQ ID NO: 62 - protein, 59 aa, Homo sapiens
KNYRSRAVSGAGWRPRSESWLCGAQRQMACGLGSHSDNGGARTQIALLTPSRTPAGIFI

SEQ ID NO: 63 - protein, 8 aa, Homo sapiens
TRLCGDNK

SEQ ID NO: 64 - protein, 18 aa, Homo sapiens
SLKQIFQVRPRSGLGVSV

SEQ ID NO: 65 - protein, 40 aa, Homo sapiens
ENAPALAVLPIYSQLPSDLQAKIFQKVCQQECPGVALGVL

SEQ ID NO: 66 - protein, 66 aa, Homo sapiens
GSGSSYSGSSSRSRSSPSPCASVSPSPSGPGSPRSSQCGLGCLPQVGWGPSGSESPWGCGRLRLKL

SEQ ID NO: 67 - protein, 136 aa, Homo sapiens
LLLPRVRGELTELERATAGAGSGSGRGRARGRGEAGLGVAERAGWGGGGEPGLGEGGGSAYLSFSIRHRSAPTRHTHKSAKPRRPPRSLIERCSGEDSVSAPPSGLPLPQFLQRPDPFSSRCPPRRGLLSPAASFL

SEQ ID NO: 68 - protein, 11 aa, Homo sapiens
LAAVLKEVCDA

SEQ ID NO: 69 - protein, 59 aa, Homo sapiens
TVLQAGKALVSGGGGGGGGRGGLGCPHVVCLPEAWLTAGPLSLRVERTVRTRRSWGMRP

SEQ ID NO: 70 - protein, 11 aa, Homo sapiens
FTAQQRQAWDR

SEQ ID NO: 71 - protein, 8 aa, Homo sapiens
KGTSLDPR

SEQ ID NO: 72 - protein, 22 aa, Homo sapiens
SSQDEVGARNHPDWVSMGGVVA

SEQ ID NO: 73 - protein, 57 aa, Homo sapiens
AAAAAVGVRAPPPIRPVAQAGPGAPVCPSPLIPWAWRWTQPSSRGQERGRCGRPWFL

SEQ ID NO: 74 - protein, 56 aa, Homo sapiens
MVSSGSREGFGGGGRSVGNSGVCRPTQGSVVSHNRSLWTERRESMGRMMNHLGVLW

SEQ ID NO: 75 - protein, 24 aa, Homo sapiens
LIPQQLLVRPAPPTPSTLSTPMPG

SEQ ID NO: 76 - protein, 21 aa, Homo sapiens
GSKHLHYWSAALPSGRDWEKR

SEQ ID NO: 77 - protein, 25 aa, Homo sapiens
FDDFSRDLCVQALLDIMDMFCDRLR

SEQ ID NO: 78 - protein, 78 aa, Homo sapiens
AKGIMQLVGQAEPESVLLVHGEAKKMEFLKQKIEQELRRQPGGRRGAHLGSARAAWAEALSSPGVQGSTATCRPMARR

SEQ ID NO: 79 - protein, 26 aa, Homo sapiens
FSGEAYEVGGVWLNVEEGVPDGLGIS

SEQ ID NO: 80 - protein, 26 aa, Homo sapiens
KEEVFEKVGVPETVLGAGLGKSASAP

SEQ ID NO: 81 - protein, 59 aa, Homo sapiens
YAGGAAQVSHRSLGKGVWERRSASAPAIGRDLPLGARYLPTAQSPKVGERASQGALGRN

SEQ ID NO: 82 - protein, 8 aa, Homo sapiens
APDPAHYR

SEQ ID NO: 83 - protein, 48 aa, Homo sapiens
RGESIYWGPTADSQDTVAGEEFPPYPTRGPRPTSRRPPPPVASLPSRP

SEQ ID NO: 84 - protein, 33 aa, Homo sapiens
AFTILAKVSVRSGPGPAGGPGCILSPCSTVSSG

SEQ ID NO: 85 - protein, 128 aa, Homo sapiens
QQDRELKVNMQTGGAGGMELVRGRTRGLCLLHIEPAGGGPGQPSWVQESFVLGVEDLYPLHPGLEGKGHHVFAWLGWVMWAPCGAWQVWSTCPDPEVQQWARPPTPTLFPWVTRGRGCPSGTPWLPDF

SEQ ID NO: 86 - protein, 68 aa, Homo sapiens
RRFIPPARCLLLLSPHSLPKASRILIKSRNRDLEPGDRDASSPSLLALSEKMYSLIPGPSPSSMDFFL

SEQ ID NO: 87 - protein, 8 aa, Homo sapiens
GPSEPTER

SEQ ID NO: 88 - protein, 10 aa, Homo sapiens
LWNQAKEVNK

SEQ ID NO: 89 - protein, 10 aa, Homo sapiens
LVMIIVRGAS

SEQ ID NO: 90 - protein, 47 aa, Homo sapiens
RCARTDKVGGGLLCPSPAAMSSTWPTFQLSVLACYAVSEHIMMYKSN

SEQ ID NO: 91 - protein, 34 aa, Homo sapiens
WQYGPCTGENPPSPTEAPLPASHPHVSEVTQALA

SEQ ID NO: 92 - protein, 57 aa, Homo sapiens
ASAADPQVRPLTLPSELPTVRHRSSPSIRPLRSAQHLPARTPTCLLPRPRSASLRPC

SEQ ID NO: 93 - protein, 8 aa, Homo sapiens
YSAPDLLG

SEQ ID NO: 94 - protein, 96 aa, Homo sapiens
EFRKHGIGKGSRARGLAPTPPPCPTPPPSHLHPVSSLAPTTLAREHPSVSSSILVPVLGQTFIIPPLPAHPPPSAILFPHAPRPFCCSLAQPPGWS

SEQ ID NO: 95 - protein, 28 aa, Homo sapiens
SDPDSDKGRSAGRGHIGGRPFLGASYEP

SEQ ID NO: 96 - protein, 28 aa, Homo sapiens
AQLMNCHVSAGTRHKVLLRRLLASFFDR

SEQ ID NO: 97 - protein, 78 aa, Homo sapiens
GRTVPSDVSPNNEYSPAGTPPPVGISSSPPWISPASMNTSHSCISLPPSCIFSLCPLISVSPWEHMPPVSGPPFFFFF

SEQ ID NO: 98 - protein, 15 aa, Homo sapiens
RINKTSPGELGQRGS

(19) United States
(12) Patent Application Publication
(10) Pub. No.: US 2021/0381060 A1
(43) Pub. Date: Dec. 9, 2021

(54) CANCER SPECIFIC IMMUNOTHERAPEUTIC TARGETS GENERATED BY CHEMOTHERAPEUTIC DRUG TREATMENT

(71) Applicants: Health Research, Inc., Buffalo, NY (US); University of Connecticut, Farmington, CT (US); Georgia State University Research Foundation, Inc., Atlanta, GA (US)

(72) Inventors: Yurij IONOV, East Amherst, NY (US); Pramod SRIVASTAVA, Farmington, CT (US); Ion MANDOIU, Farmington, CT (US); Alex ZELIKOVSKY, Atlanta, GA (US)

(21) Appl. No.: 17/287,448

(22) PCT Filed: Oct. 29, 2019
(86) PCT No.: PCT/US19/58582

§ 371 Date: Apr. 21, 2021

Related U.S. Application Data
(60) Provisional application No. 62/752,149, filed on Oct. 29, 2018.

(51) Int. Cl. C12Q 1/6886; A61K 39/00; A61K 31/513; C12N 15/10; A61P 35/00

(52) U.S. Cl. CPC C12Q 1/6886; A61K 39/001151; C12Q 2600/136; C12N 15/1093; A61P 35/00; A61K 31/513

(57) ABSTRACT

Provided are methods for identifying antigens containing amino acid sequences for use in a cancer vaccine. The vaccines and methods of use for prophylaxis and/or therapy of cancer are included. The method involves: i) exposing cancer cells to a chemotherapeutic agent that damages DNA; ii) determining open reading frames encoded by mRNA transcribed from a gene in the cancer cells of i); iii) comparing the open reading frames of the mRNA of i) to open reading frames encoded by mRNA transcribed from the gene in the cancer cells that were not exposed to the chemotherapeutic agent, iv) determining a different open reading frame encoded by the mRNA of i) and an open reading frame of the mRNA of ii), wherein the different open reading frame encoded by the mRNA of i) encodes a contiguous amino acid sequence comprising the sequence of the antigen for use in the cancer vaccine.

### CROSS-REFERENCE TO RELATED APPLICATIONS

This application claims priority to U.S. provisional application no. 62/752,149 filed on Oct. 29, 2018, the disclosure of which is hereby incorporated by reference.

### FIELD

The present disclosure relates to methods and compositions for identifying antigens for use in cancer vaccine formulations.

### BACKGROUND

The tumor suppressor p53 is mutated or lost in about half of all human cancers. Loss of p53 function is known to influence cell cycle checkpoint controls, thus enabling p53-deficient tumor cells with DNA damage to continue cycling, allowing the acquisition of additional mutations contributing to metastasis. Gene expression profile alterations that occur in p53 mutant cells in response to DNA damage differ from those in the cells with wild type p53. As DNA damage is known to affect splicing of multiple genes and given that in cancer, the splicing process is commonly disrupted, the pattern of aberrant splicing in cells with mutant p53 differs from that of the cells with the wild type p53.

Tumor suppressor p53 is the transcription factor which is activated in response to DNA damage. It works not only to activate transcription of target genes but also to repress the transcription of a number cell cycle checkpoint genes that promote cell proliferation. Chemotherapeutic drug 5-fluorouracil (5-FU), as well as other chemotherapeutic agents, is known not only to cause DNA damage, but also to inhibit splicing of pre-mRNA resulting in aberrant splicing, particularly in retention of introns. There is an ongoing and unmet need to exploit this phenomenon to provide improved compositions and methods for treating cancer. This present disclosure is pertinent to this need.

### SUMMARY

The present disclosure provides methods and compositions that relate generally to identifying cancer-specific antigens for use in cancer vaccine formulations. Chemotherapies, such as 5-fluorouracil, induce aberrant RNA splicing resulting in unique, cancer-specific RNA molecules known as Chemotherapy-induced Products of Aberrant Splicing (CiPAS) which produce unique proteins that can be used as antigens in anti-cancer vaccines. In embodiments, CiPAS and chemo-neoepitopes are present in cancer cells in an individual. In embodiments, production of CiPAS provides for identifying and using polypeptides that are capable of generating humoral and/or cellular immune responses against neoantigens or neoepitopes that are expressed only in cancer cells. In embodiments, the CiPAS and/or neoantigens are identifiable in any cancer that has been treated with a chemotherapeutic agent.

In embodiments, the disclosure provides a vaccine composition comprising a pharmaceutical formulation comprising CiPAS antigens, as further described herein.

Aspects of this disclosure are demonstrated in the detailed description, and in particular, in Example 2 and FIG. 3. This example proves that treatment of tumor cells with fluorouracil renders tumor cells immunogenic against an antigenically un-related tumor.

In embodiments, the disclosure provides method for identifying antigens comprising amino acid sequences for use in a cancer vaccine. In embodiments, the method comprises:

i) exposing cancer cells to a chemotherapeutic agent that damages DNA;

ii) determining open reading frames encoded by mRNA transcribed from a gene in the cancer cells of i);

iii) comparing the open reading frames of the mRNA of i) to open reading frames encoded by mRNA transcribed from the gene in the cancer cells that were not exposed to the chemotherapeutic agent,

determining a different open reading frame encoded by the mRNA of i) and an open reading frame of the mRNA of ii), wherein the different open reading frame encoded by the mRNA of i) encodes a contiguous amino acid sequence comprising the sequence of the antigen for use in the cancer vaccine. In embodiments, the open reading frames of the mRNA of i) are encoded by a sequence from an improperly spliced mRNA, such as an mRNA comprising an intron or a segment of a retained intron. In embodiments, the method may be repeated using a plurality of distinct cancer cells to determine a plurality of distinct antigen sequences. In embodiments, the cancer cells comprise a mutated p53 protein. In embodiments, the cancer cells are human cancer cells. In certain approaches, the method also may comprise producing a peptide comprising a contiguous amino acid sequence comprising the sequence of the antigen. In embodiments, the peptide comprises, or consists, of from 9-11 contiguous amino acids selected from the amino acid sequences elected from the amino acid sequence presented in Table 1. In embodiments, the disclosure provides for mixing the produced peptide with a pharmaceutically acceptable agent. In embodiments, the combination of the pharmaceutically acceptable agent provides a pharmaceutical formulation, such as a vaccine formulation. In embodiments, the vaccine formulation comprises at least one pharmaceutically acceptable adjuvant, which may enhance the immune response stimulated by the vaccine, relative to a formulation that does not comprise the adjuvant.

In one aspect, the disclosure comprises administering a vaccine formulation produced according to the disclosure to an individual in need thereof. In embodiments, this method further comprises administering to the individual a chemotherapeutic agent that damages DNA such that a polypeptide comprising the sequence of the antigen is produced by cancer cells in the individual. In certain and non-limiting approaches, the individual has not previously been treated with the chemotherapeutic agent before administering the vaccine. In embodiments, the peptide identified and produced according to this disclosure comprises, or consist, of a peptide having 9-11 contiguous amino acids selected from the amino acid sequences presented in Table 1.

Expression vectors encoding a polypeptide comprising the amino acid sequence of a peptide identified by the method of the disclosure are included. The disclosure also includes a plurality of peptides identified by the method of the disclosure, such as a library of peptides that can be used as off the shelf vaccines. The off the shelf vaccines may be suitable for any particular Human leukocyte antigen (HLA) type of an individual to be treated with a vaccine formulation as described herein.

### BRIEF DESCRIPTION OF THE FIGURES

FIG. 1. Action of 5-FU and other selected agents leads to DNA damage and aberrant splicing. These two effects induce a number of events that are specific to cancer cells and lead to generation of CiPAS, specifically in cancer cells, secondarily leading to translation of CiPAS and generation of chemo-neoepitopes that are the same in all cancer cells and are specific to cancer cells.

FIG. 2. Intron retention induced by 5-FU treatment of p53 mutant but not p53 wild-type cells. P53 mutant MDA-MB-231 cells, p53wt MCF10A cells, and MCF10A cells with biallelic frameshift mutation in the p53 gene were treated or untreated with 300uM of 5-FU for 24 h. NMD was inhibited or not with 10 mM caffeine for 4 h before cytoplasmic RNA isolation. “*” symbols indicate RT-PCR amplicons with retained introns.

FIG. 3. BALB/cJ mice were challenged (on the left flank) at day 0 with 150,000 5FU- treated or untreated 4T1 tumor cells. Seven days after the first tumor challenge, they were challenged (on the right flank) with 95,000 5FU- treated or untreated Meth A tumor cells and growth of both tumors was measured. A. Un-treated or 5FU-treated Meth A cells grow similarly in vivo (P=0.07). 5FU treatment by itself does not influence tumor growth kinetics. Each line represents tumor growth in a single mouse. Right panel displays area under the curve (AUC) for both groups. B. (Top panel) 4T1 (un-treated or in vitro-5FU treated) growth curves in 5FU-treated mice (5FU i.p. 100 mg/kg at day -7, 50 mg/kg at day 0 and 20 mg/kg every three days after the first tumor challenge). Right panel displays AUC. 5FU treated and un-treated 4T1 cells grow the same in 5FU-treated mice (P=0.69). (Bottom panel) The same mice as the top panel were challenged with un-treated or 5FU-treated Meth A cells, one week after challenge with 4T1 cells. The AUC value for Meth A growth curves in mice challenged with “5FU treated Meth A cells” is significantly lower than that of mice challenged with “untreated Meth A cells” (P=0.003).

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein. Each RNA sequence includes its DNA equivalent, and each DNA sequence includes its RNA equivalent. Complementary and anti-parallel polynucleotide sequences are included. All polynucleotide and amino acid sequences described or otherwise referenced herein include homologous, and variant. Sequences having from 50-99% similarity, including but not limited to such similarity across the entire length of such sequences, are included in this disclosure.

Although subject matter of this disclosure will be described in terms of certain embodiments, other embodiments are also within the scope of this disclosure. Various changes may be made without departing from the scope of the disclosure.

Each gene described herein may be known in the art, although abnormally spliced mRNA from such genes, and translation of proteins from the abnormally spliced mRNA, in response to chemotherapeutic is believed to be a novel aspect of this disclosure.

All compositions of matter described herein can comprise or consist of any one or combination of composition components, and all steps may comprise or consist of the described steps. The steps may be performed sequentially, and one or more steps may be omitted.

In embodiments, the disclosure relates to methods of identifying amino acid sequences for use in vaccines. The amino acid sequences are comprised by or consist of proteins, or fragments or segments of such proteins, wherein the proteins are translated from mRNA that has been aberrantly spliced due to exposure of genetic material to one or more chemotherapeutic agents. “Aberrantly” spliced mRNA means abnormal mRNA that is not completely or correctly spliced, i.e., mRNA that contains at least some portion of one or more introns that were present in the heteronuclear RNA, prior to splicing to produce mRNA, or exons that are not correctly joined to one another. Thus, the cytoplasmic mRNA may comprise one or more introns, or fragments of introns. The mRNA may therefore be fully unspliced, or may be underspliced, i.e., one or more introns or segments thereof have been spliced out of the mRNA, but at least an intron or a segment of an intron remains in the aberrantly spliced mRNA. In embodiments, the abnormal splicing result in junction of a part of the exon with another exon or part of the other exon resulting in alternative reading frame coding for a novel amino acid sequence. In embodiments, retained introns or incorrectly joined exons result in an altered or disrupted open reading frame (ORF). The altered or disrupted ORF may have, for example, an abnormal translation site (a stop codon), and/or may introduce additional encoded amino acids, wherein a normally spliced mRNA would not encode such amino acids. Thus, proteins translated from aberrantly spliced mRNA may be truncated, or longer, or have insertions, deletions, mutations, including but not limited to missense and nonsense mutations, or combinations thereof, relative to their wild type counterparts.

In related embodiments, a DNA vaccine that encodes an amino acid sequence encoded by a retained intron or segment thereof or by having an alternative reading frame of the exon is identified using a method described herein, and can further include producing such DNA vaccines, and/or the proteins and/or peptides encoded by the DNA. Thus, in embodiments, the disclosure provides isolated polypeptides and/or peptides, and vaccine formulations, and DNA vaccines including such polypeptides and/or polypeptides, and methods of using the same, to stimulate an immune response against segments of the polypeptides that are encoded in whole in part by an intron or segment thereof that is retained in the mRNA or by alternative reading frame of the abnormally spliced exon.

In embodiments, an antigen that is comprised by a protein translated from an abnormally spliced mRNA is referred to herein as a “neoantigen” which comprise “chemo-neoepitopes” as described further below.

In embodiments, the disclosure comprises assessing differences in biological samples pre- and -post chemotherapy to determine neoantigens and neoepitopes that are expressed as a result of exposure to a chemotherapeutic agent. In embodiments, the disclosure comprises testing a biological sample, which may be a patient sample or a sample derived from a patient sample, or a cell line, and may be a sample of normal (non-cancerous) tissue, and a sample of comprising malignant cells (cancer cells), subjecting one or both samples to a process, such as exposure to one or a combination of chemotherapeutic agents, and determining a difference in mRNA and/or protein expression in the cancer sample, analyzing the cancer sample relative to the normal sample or any other suitable control, to identify mRNA and/or proteins that are differently expressed in the cancer sample relative to the normal sample or other suitable control.

Chemotherapeutic agents that induce DNA damage are known in the art. In general, the chemotherapeutic agents referred to herein will induce a DNA damage response. In embodiments, the chemotherapeutic agents include but are not necessarily limited to alkylating agents, platinum-based agents, DNA intercalating agents, topoisomerase poisons or other replication disrupting agents, radiomimetics, anti-metabolites, and combinations thereof.

In embodiments, alkylating agents include but are not limited to Bendamustine and Melphalan. In embodiments, platinum-based agents include but are not limited to Cisplatin, Carboplatin, and Oxaliplatin. In embodiments, anti-metabolites include but are not limited to 5-fluorouracil. In embodiments, the chemotherapeutic agent may be gemcitabine, methotrexate or bleomycin. In embodiment, the chemotherapeutic agent is any agent that is known to inhibit splicing of pre-mRNA. In embodiments, the cancer cells analyzed according to methods of this disclosure have abnormally functioning p53 protein. In embodiments, abnormally functioning p53 comprises a mutation. In embodiments, the p53 mutation results in p53 protein that has lost tumor suppressor function, and/or gained dominant negative function, and/or exhibits oncogenic function. In non-limiting embodiments, the p53 mutations comprise one or a combination of common cancer-associated p53 mutants, such as R175H, R248Q, R273H, R280K or E285A, but many more are known in the art and apply to this disclosure.

The disclosure further comprises designing and/or producing recombinant expression vectors that encode the differently expressed proteins, or segments thereof that are encoded by one or more retained introns, or are produced from an mRNA that contains one or more retained introns or by alternative reading frame due to an abnormally spliced exon(s). The disclosure further comprises expressing and separating the differently expressed proteins, or segments thereof, from an expression system. The disclosure further comprises formulating the differently expressed proteins, or segments thereof, into pharmaceutical formulations for use in methods of prophylaxis and/or therapy of cancer patients, which may be in conjunction with chemotherapeutic treatment. The disclosure further comprises administering the vaccines to an individual in need thereof. Vaccine administrations can be performed prior to, concurrently, or subsequent to the chemotherapeutic treatment. In embodiments, the vaccine is administered prior to chemotherapeutic treatment. In embodiments, a DNA vaccine can be used. In embodiments, the vaccine composition is administered to an individual prior to undergoing chemotherapy and/or after undergoing chemotherapy. In additional embodiments the vaccine composition can be administered intravenously, intradermally, intramuscularly, mucosally, and/or orally, including by inhalation or intranasal application.

For any vaccine described herein where a therapeutically effective amount is not established, the therapeutically effective amount, e.g., a dose, can be estimated initially either in cell culture assays or in animal models. Such information can then be used to determine useful doses and routes for administration in humans. A precise dosage can be selected by the individual physician in view of the patient to be treated. Dosage and administration can be adjusted to provide sufficient levels of the active moiety or to maintain the desired effect. Additional factors which may be taken into account include the severity and type of the cancer, age, weight and gender of the patient, desired duration of treatment, method of administration, time and frequency of administration, drug combination(s), reaction sensitivities, and tolerance/response to therapy. In embodiments, administering a therapeutically effective amount of vaccine composition produces at least one of the following results: reduction or eradication of cancer cells and/or a tumor in the individual; a reduction in tumor size and/or an inhibition of tumor growth; and inhibition of metastasis; reduced occurrence or prevention of relapse into chemotherapeutic drug resistance; an improved prognosis for the cancer; or an extended life span for the cancer patient.

Peptides of the invention can be prepared by any technique known to those skilled in the art or by techniques hereafter developed. “Peptide” and its plural forms refer to short polypeptides, such as those ranging in size of from 8-30 amino acids, inclusive, and including all ranges of numbers there between. In embodiments, the peptides comprise or consist of from 9-11 amino acids. Peptides of this disclosure can be prepared using any suitable approach, non-limiting examples of which include the solid-phase synthetic technique (Merrifield, J. Am. Chem. Soc., 15:2149-2154 (1963); M. Bodanszky et al., (1976) Peptide Synthesis, John Wiley & Sons, 2d Ed.; Kent and Clark-Lewis in Synthetic Peptides in Biology and Medicine, p. 295-358, eds. Alitalo, K., et al. Science Publishers, (Amsterdam, 1985). The synthesis of peptides by solution methods may also be used, as described in The Proteins, Vol. II, 3d Ed., p. 105-237, Neurath, H., et al., Eds., Academic Press, New York, N.Y. (1976). The synthesized peptides may be substantially purified by preparative high performance liquid chromatography or other comparable techniques available in the art. The composition of the synthetic peptides can be confirmed by any technique for amino acid composition analysis.

The peptides of the present invention may be formulated with a suitable adjuvant in order to enhance the immunological response. Suitable adjuvants include but are not limited to mineral salts, including aluminium hydroxide and aluminium and calcium phosphate gels, oil emulsions and surfactant based formulations, saponin, AS02 [SBAS2] (oil-in-water emulsion), Montanide ISA-51 and ISA-720, particulate adjuvants, including virosomes, AS04, [SBAS4] Al salt with MPL, ISCOMS (structured complex of saponins and lipids), polylactide co-glycolide (PLG), natural and synthetic microbial derivatives, lipoidal immunostimulators OM-174 (lipid A derivative), synthetic oligonucleotides containing immunostimulatory CpG motifs, modified bacterial toxins, endogenous human immunomodulators, including hGM-CSF and hIL-12, hIL-15, hIL-17, hIL-21, Immudaptin and inert vehicles, including gold particles. The peptides can be administered in a conventional dosage form prepared by combining the peptides with a standard pharmaceutically acceptable carrier according to known techniques. Some examples of pharmaceutically acceptable carriers can be found in: Remington: The Science and Practice of Pharmacy (2005) 21st Edition, Philadelphia, Pa. Lippincott Williams & Wilkins.

In one embodiment, the peptides of the invention may be conjugated to an immunogenic carrier protein. Suitable carriers include but are not limited to Limulus polyphemus hemocyanin (LPH), Tachypleus tridentatus hemocyanin (TTH), and bovine serum albumin (BSA), tetanus toxoid and diphtheria toxin, DHBcAg, polyribotol ribosyl phosphate (PRP), PncPD11, and nanoparticle formulations. In one embodiment, a suitable immunogenic carrier protein is Keyhole Limpet Hemocyanin (KLH).

The peptides of the invention may also be administered as peptide loaded with antigen presenting cells (APCs), such as dendritic cells or macrophage. Thus, the method includes administering to the individual APCs that have been incubated with a peptide of the invention such that the APCs cells have taken up the peptide to obtain peptide loaded dendritic cells that facilitate of HLA presentation epitope(s) present in the peptide. The APCs employed for this purpose may be isolated from the individual to whom they are to be delivered after incubation with the peptide, or they may be obtained from an allo-matched individual. Accordingly, the invention also provides a composition comprising a substantially purified population of APCs, wherein the APCs have been incubated with a peptide of the invention such that the APCs cells take up and display the neo-antigen.

In embodiments, treatment of a patient with a vaccine formulation can be combined with other interventions, including standard chemotherapeutic treatments, immune checkpoint inhibitors or other antibody-based agents, and surgical interventions.

In embodiments, any peptides described in this disclosure are suitable for use with many human HLA types. Representative and non-limiting embodiments of CiPAS are described in Table 1. The disclosure includes contiguous fragments of from 9-11 amino acids of any segment of the CiPas. Data obtained by the inventors indicate that such segments of polypeptides are suitable for use with of the most common human HLA alleles. In embodiments, the HLA alleles comprise HLA-II alleles.

In embodiments, a peptide of this disclosure may be modified, such as by being included in a contiguous polypeptide that comprises other amino acid sequences, such as sequences used for purification, or to improve bioavailability, to increase its presence or duration in an HLA cleft, etc.

It will be recognized from this disclosure that any result obtained herein can be fixed in a tangible medium of expression, and/or in one or more digitized files. In embodiments, the disclosure provides an index or library of distinct neoantigens and/or neoepitopes. In embodiments, the library comprises recombinantly expressed and/or modified peptides or proteins. In embodiments, the disclosure includes testing a sample from an individual, and based on one or more characteristics of the sample, administering to the individual a vaccine as described herein. Thus, the disclosure is adaptable for personalize medicine approaches.

In embodiments, the disclosure provides for identifying and using polypeptides that are capable of generating humoral and/or cell-mediated immune responses to neoantigens and/or neoepitopes that are only expressed in cancer cells. In non-limiting approaches, the disclosure provides for identifying and using polypeptides that can generate cytotoxic or helper or regulatory T-cells against continuous amino acid sequences encoded by retained introns or by alternative reading frames of the exons produced by abnormal splicing, or encoded by an mRNA that contains retained introns or alternative reading frames, despite the degradation of aberrantly spliced mRNA through the nonsense mediated decay (NMD) pathway.

In embodiments, the disclosure provides compositions and methods that relate to stimulating an immune response against antigens that are encoded in whole or in part by retained introns, or segments thereof. In embodiments, and as described above, the disclosure further comprises identification of such antigens by testing biological samples from an individual for the presence of aberrantly spliced mRNA, and/or for the presence of proteins that comprise amino acids encoded by retained introns or segments thereof. In certain embodiments, the method further comprises immunization against the identified antigenic sequences, such as after surgical removal of a primary tumor. In embodiments, after induction of an immune response against the products of abnormal splicing using polypeptides identified according to methods described herein, the disclosure comprises treating cancer patients with one or more chemotherapeutic drugs that generate corresponding products of aberrant splicing in cancer cells that contain a mutant p53 gene, and thereby express a mutated p53 protein or do not express p53 protein at all or at a detectable level, but wherein cells with a normal (i.e., non-mutated or wild type) p53 gene do not express the products of aberrantly spliced mRNA. The disclosure therefore provides an ongoing, endogenous supply of antigens that can further stimulate the immune system.

In embodiments, the strategy provided by the present disclosure is expected to facilitate generation of strong immune response against novel cancer specific antigens that are not expressed in normal (non-cancer) cells. Aspects of this disclosure are demonstrated in Example 2, which shows that treatment of tumor cells with fluorouracil renders tumor cells immunogenic against an antigenically un-related tumor.

In embodiments, chemotherapeutic treatment of cancer patients immunized in advance against proteins that occur in cancer patients in response to chemotherapy is expected to be more efficient than chemotherapy or immunotherapy alone. In embodiments, practicing a method of this disclosure provides for use of chemotherapy and/or immunotherapy against cancer cells that can efficiently eliminate metastatic cells. Thus, in certain aspects, a vaccine of this disclosure is administered to a cancer patient prior to a first administration of a chemotherapeutic drug, such as a chemotherapeutic drug that can induce in whole in part production of aberrantly spliced mRNA, and/or affect the activity of p53. In embodiments, vaccine of this disclosure is administered to a cancer concurrent with or subsequent to a first administration of a chemotherapeutic drug. Accordingly, the disclosure provides off the shelf cancer vaccines, representing both prophylactic and therapeutic cancer treatment modalities.

The type of cancer against which the presently provided compositions may stimulate an immune response is not particularly limited, provided the cancer cells translate a protein from an aberrantly spliced mRNA, the expression of which is induced in whole or in part by a chemotherapeutic agent. In embodiments, the cancer comprises a tumor. In embodiments, the cancer is a liquid cancer. In embodiments, the cancer is breast cancer, prostate cancer, colon cancer, brain cancer, lung cancer, pancreatic cancer, skin cancer including but not limited to melanoma, stomach cancer, head and neck cancer, mouth cancer, esophageal cancer, bone cancer, ovarian cancer, colon cancer, uterine cancer, endometrial cancer, testicular cancer, bile duct cancer, bladder cancer, laryngeal cancer, thyroid cancer, retinoblastoma, any sarcoma and any carcinoma. In embodiments, the individual has blood cancer, including but not limited to any leukemia, lymphoma, or myeloma.

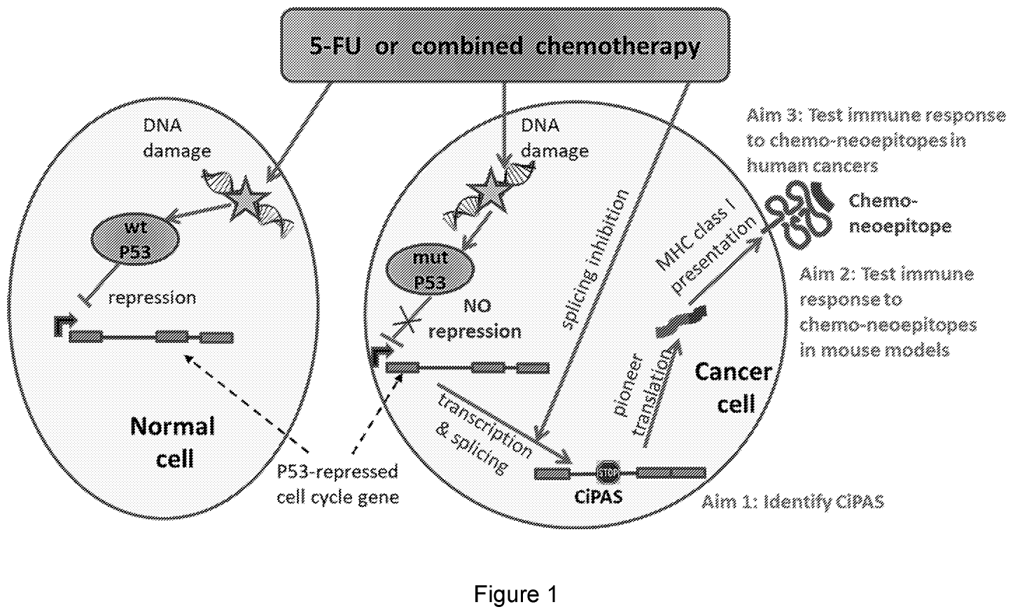

Without intending to be bound by any particular theory, it is considered that the present disclosure encompasses certain molecular and cell biology principles, as generally outlined in FIG. 1. First, cells experiencing DNA damage repress transcription of several cell cycle genes. Second, cells with mutated p53 genes are unable to mediate this transcriptional repression, creating a clear transcriptional difference between normal and cancer cells within a host. Third, certain chemotherapies (e.g., platinums, 5 Fluorouracil, etc., and as further described herein), in addition to causing DNA damage, cause abnormal splicing leading to generation of abnormal transcripts that are recurrent and reproducible in cells treated with such chemotherapy. Fourth, the abnormal splicing products undergo Nonsense Mediated mRNA Decay, but not before a Pioneer Round of translation, which creates antigenic epitopes.

Without intending to be constrained by any particular theory, the present approach is based at least in part on the discovery that the aberrant transcripts induced by chemotherapy (referred to herein for convenience as “Chemotherapy-induced Products of Abnormal Splicing” or “CiPAS”) are expressed reproducibly in all cancer cells treated with the same chemotherapy. While undergoing translation, and as described above, CiPAS create aberrant polypeptides which include novel amino-acid sequences encoded by retained introns, or by improperly spliced exons. These aberrant polypeptides, parts of which are not translated in cells not exposed to chemotherapy, are the substratum for generation of neoepitopes, i.e., the chemo-neoepitopes, because they are generated only in response to chemotherapy. It is believed these chemo-neoepitopes have not previously been subjected to mechanisms of central deletion or peripheral tolerance. Stated differently, they are foreign to the immune system of any particular individual, and hence, are considered to represent ideal immunogens.

Again, without intending to be constrained by theory, it is considered that CiPAS and chemo-neoepitopes are specific to cancer cells because of another property of these selected chemotherapies—that they induce DNA breaks which are sensed by wild type p53 leading to suppression of cell cycle genes via the p53-DREAM pathway. This phenomenon does not occur in cancer cells which lack a functional p53, hence, the cancer-specificity of CiPAS and chemo-neoepitopes. Accordingly, in certain approaches, the present disclosure is based at least in part on the approach that: (1) Selected chemotherapies lead to generation of CiPAS that are not expressed in untreated cells or in treated cells with a functional p53, and (2) CiPAS create new and predictable chemo-neoepitopes, immunization with which will contribute to tumor eradication.

In an embodiment, the disclosure provides a platform to identify CiPAS which are recurrently produced during chemotherapy that harbor p53 mutations. The disclosure is illustrated using breast cancer tumors, but is expected to be adaptable to any cancer that produces aberrantly spliced mRNA in response to a chemotherapeutic agent, including but not necessarily limited to any chemotherapeutic agent that induced DNA breaks, such as double stranded DNA breaks. In one approach, bulk RNA-Seq and both bulk and single cell RT-PCR is used to validate CiPAS specifically and recurrently expressed in tumors. In a non-limiting demonstration, aspects of the disclosure use p53^{-/-} breast tumors. In embodiments, all tumor samples used to illustrate a non-limiting aspect of the disclosure may either be deficient in p53 naturally or rendered so by transfection with dominant negative p53: 4T1 mouse line; human lines MDA-MB-231, MCF10A cells, triple negative breast cancer tissues treated with carboplatin/other therapies. In related embodiments, the disclosure provides for determining CiPAS that generate cancer-specific chemo-neoepitopes that elicit immune protection from tumor growth of triple negative breast cancers. In one non-limiting approach, the disclosure includes identification of chemo-neoepitopes generated from CiPAS in chemotherapy-treated mouse breast cancer line 4T1 (p53−/−) but not in EMT6 (p53+/+) by using RNA-Seq, mass-spectrometry and an integrative bioinformatics approach.

The disclosure includes testing chemo-neoepitopes for their ability to elicit CD8+ and CD4 T+ cells and tumor rejection using p53−/− 4T1 triple-negative breast cancer line or as negative control, p53+/+ EMT6 breast cancer line in syngeneic BALB/c mice. In related and also non-limiting embodiments, the disclosure provides for identification of CiPAS in cancer tissues from platinum-based chemotherapy-treated breast cancer patients who have received neoadjuvant chemotherapy, and characterizing them for expression of cell cycle genes including but not necessarily limited to CDC25A, CCNA2, CHEK1, NEK2, CDC20, and CDC6. Primary cultures can be tested before/after chemotherapy in vitro.

In a related and non-limiting aspect, the disclosure provides for predicting CiPAS-encoded chemo-neoepitopes based on HLA alleles of individual patients, and analysis of blood and/or tumor-infiltrating lymphocytes for T cell responses to them.

In general, to identify CiPAS which are recurrently produced during chemotherapy in breast tumors that harbor p53 mutations, the disclosure provides libraries of abnormal splicing products (CiPAS) which enable production of a novel class of therapeutic anticancer vaccines. The CiPAS have the following characteristics: (i) they are expressed exclusively in response to chemo-therapy treatment and exclusively in cancer cells with p53 gene mutations, (ii) are shared by different p53 mutant tumors of the same tissue type, and (iii) encode novel peptide sequences that contain MHC class I epitopes. If desired, candidate CiPAS can be generated by bioinformatic analysis of bulk RNA-Seq from breast cancer cell lines and tumor tissues, and validated in a larger panel by bulk and single cell RT-PCR.

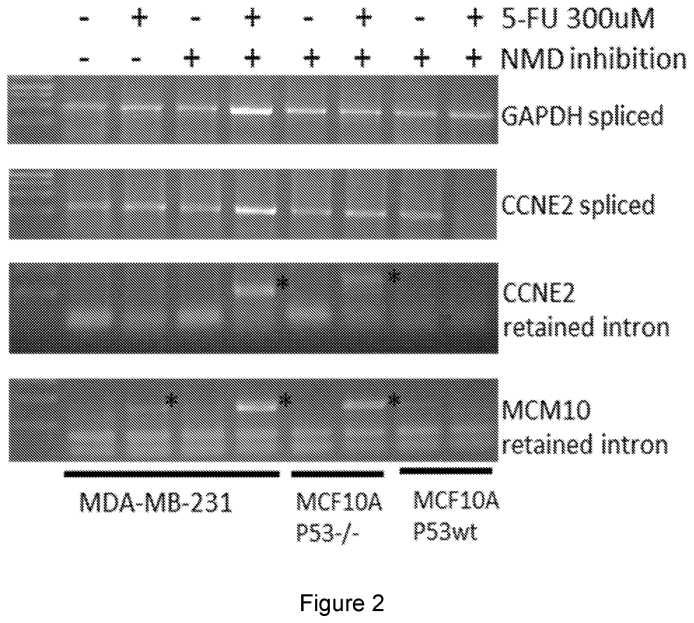

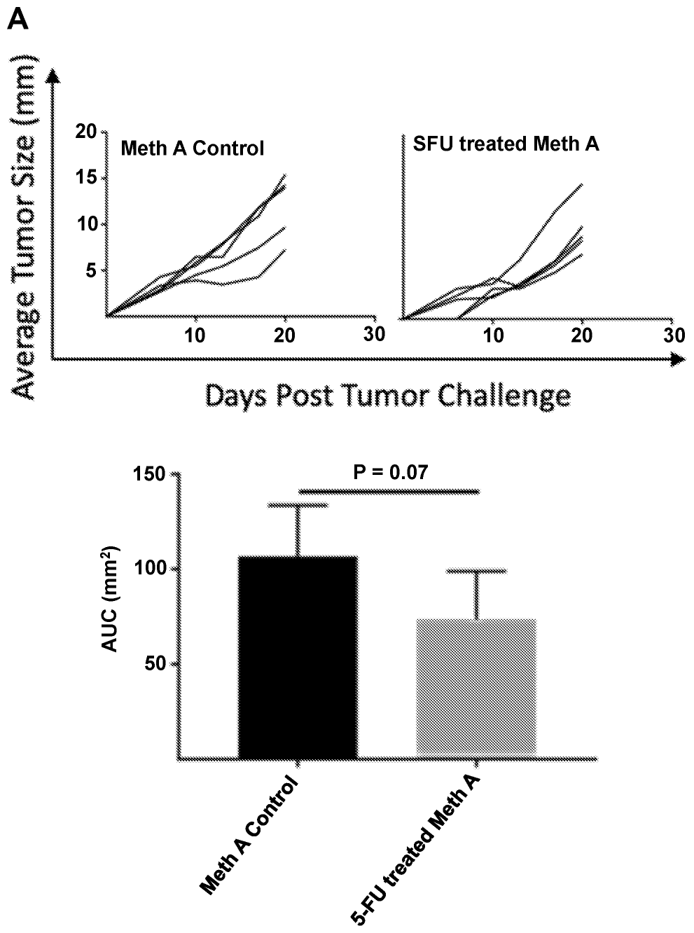

In arriving at the present disclosure, we analyzed cancer specific intron retention events in several cell cycle progression-related genes in response to 5-FU exposure in human breast cancer cell lines. The following breast cell lines were used: MDA-MB-231 p53−/−, MCF10A p53+/+ cell line and the derivative of MCF10A p53−/−, all treated or untreated with 300 μM of 5-FU for 24 h. In addition, the NMD pathway was inhibited or not inhibited with caffeine for 4 h before cytoplasmic RNA isolation in order to detect the mRNA species that are degraded due to stop codons contained within retained introns. FIG. 2 shows that the transcription of the mRNA of the CCNE2 cell cycle progression gene is repressed in response to 5-FU only in MCF10A cells with p53+/+. The MCM10 mRNA was also repressed (not shown). On the other hand, the RT-PCR analysis demonstrates the intron retention in MCM10 and CCNE2 genes in response to 5-FU only in cells with mutant p53. Importantly, the same introns are retained in the same genes in response to 5-FU treatment of different cell lines with different p53 mutations, although only part of the 6th intron of the CCNE2 gene is retained in the MDA-MB-231 cells, whereas the whole intron is retained in MCF10A p53−/− cells. To assess the extent of intron retention induced by 5-FU treatment we performed whole transcriptome sequencing of 4T1 cells treated or untreated with 5-FU (300 uM for 24 h before RNA isolation) and caffeine (10 mM for 4 hours before RNA isolation), and of spleen and intestine tissues of mice treated or untreated with 5-FU (150 mg/kg of mouse body weight). Each of the 8 samples was sequenced at a depth of ˜100M reads (range 96M-154M) using 2×150 bp stranded RNA-Seq. Analysis of the RNA-Seq data using the IRFinder package identified 991 introns in 804 genes overexpressed (at a p-value cutoff of 0.001) in the 4T1 samples treated with 5-FU (with or without caffeine) compared to 4T1 samples not treated with 5-FU and the normal tissues. Several of these retained introns have been validated by RT-PCR/Sanger sequencing, including intron 4 of the Ppp1r13l gene, which encodes the strong H2-Kd binder having the amino acid sequence Ser, Tyr, Thr, followed by Leu, Ile, His, followed by Gly, Pro, Leu, in the N- to C- terminal direction. Notably, the protein phosphatase 1 regulatory subunit 13 like encoded by Ppp1r13l is one of the most evolutionarily conserved inhibitors of p53, with which has been hypothesized to form a regulatory feedback loop controlling genotoxic stress responses.

Cells and tissues. Mouse breast cancer cell lines 4T1 and EMT6 are used. The 4T1 cells are null for p53 expression while EMT6 is p53+/+ and is used as a negative control. Human cell lines and tissues may also be used: triple negative breast cancer cell line MDA-MB-231 which is p53 deficient, a derivative of MCF10A cells which is also p53 deficient, can be use. P53-proficient lines can be used as controls. In addition, core biopsies of triple negative breast cancer human tissues (from patients who are to receive neoadjuvant chemotherapy—Adriamycin, Cytoxan, Taxotere with or without Carboplatin) can be obtained, as will samples from these patients post-surgical resection.

RT-PCR analysis to identify preferable drug or drug combinations for generating recurrent CiPAS. Drug concentrations for generating CiPAS can be determined. In a non-limiting embodiment, the optimal concentration can be the minimal concentration that (i) completely represses the mRNA expression of cell cycle-related genes (including but not limited to NEK2, CDC6, CCNE2, MCM10 and other MCM family members) in p53+/+ cells and (ii) induces a high enough number of abnormal splicing events generating CiPAS encoding chemo-neoepitopes.

Tumor-specific CiPAS identification by de novo RNA-Seq sequencing analysis. The optimal concentration of each drug can be used to treat cells for isolation of cytoplasmic RNA from the drug treated/untreated p53−/− and p53+/+ cells. Two p53−/− and two p53+/+ lines can be used for identifying recurrent CiPAS using RNA-Seq. The CiPAS that occur in cell lines with mutant p53 but not in cell lines with functional p53 are likely to be recurrent events and to occur in other p53−/− cancers of the same tissue type. The cells for the RNA-Seq analysis can be treated or not treated with caffeine in order to inhibit or not to inhibit the NMD in the drug-treated cells, respectively. We have demonstrated that analyzing global RNA level alterations induced by NMD inhibition with caffeine treatment one can identify mRNA transcripts containing premature translation termination codons. The CiPAS that do not trigger NMD but occur exclusively in cells with mutant p53 can also be analyzed. Due to a high level of expression the peptides from such products can be highly represented by HLA-class I. By comparing the RNA-Seq mRNA expression levels from caffeine treated and untreated cells, CiPAS that are degraded through the NMD can be distinguished from CiPAS that are not.

Two complementary bioinformatics approaches to identify CiPAS from RNA-Seq data can be used. In the first, annotation-guided approach, reads are trimmed for adapters, mapped to the genome using spliced aligners with high sensitivity settings, and then tested for evidence of differential intron retention and other alternative splicing events using statistical tests appropriate for digital expression data with small number of replicates. For intron retention, coverage-based approaches such as IRFinder, which have been found to produce reliable results at lower sequencing depth can be used. Other alternative splicing events can be detected by junction coverage analysis performed using VAST-TOOLS. To ensure robustness, bootstrapping and read subsampling analysis can be used. Differential expression between samples treated with both 5-FU and caffeine and those treated with 5-FU alone can be used to classify the identified CiPAS as targeted by NMD or not.

Since chemotherapy may induce completely novel splicing events not detected by the annotation-guided approach, a de novo approach to RNA-Seq data analysis can be employed, such as by using suitable software, such as Trinity software, to assemble trimmed RNA-Seq reads and identify novel splicing events that occur in the p53−/− but not in p53+/+ cells. Trinity uses a de Bruijn graph-based methodology for de novo transcriptome reconstruction from RNA-Seq reads and has been shown to reconstruct a large fraction of the transcripts present in the data, including alternative splice isoforms. Although Trinity can be used in the absence of a reference genome, it can take advantage of the reference when available. To identify retained introns with coding potential, open reading frames (ORFs) predicted from the Trinity transcript sequences can be translated to proteins and searched against the Uniprot mouse protein database. BLAST matches shorter than translated ORFs by more than 10 amino acids can be analyzed for evidence of retained introns or cryptic exons.

Verification of recurrent CiPAS using RT-PCR analysis. RT-PCR analysis is used to verify that the events also occur in response to drug treatment in other cancer cell lines with p53 mutations. These can include breast cancer cell lines from the ATCC Breast Cancer p53 Hotspot Mutation Cell Panel which include AU565 (R175H), SK-BR-3 (R175H), HCC70 (R248Q), BT-549 (R249S), HCC38 (R273L), and MDA-MB-468 (R273H), and other cancer cells that will be apparent to those skilled in the art, given the benefit of the present disclosure. The recurrent CiPAS can be analyzed by RT-PCR of both bulk and single-cell cytoplasmic RNA to confirm specific expression in p53 mutant cells as well as lack of retention of preceding introns which can introduce upstream stop codons. A product of this analysis comprises a list of CiPAS along with the novel amino sequences.

Identifying cancer specific CiPAS in mouse cancer cells. CiPAS that arise in mouse 4T1 p53-null breast cancer cells from genes that are transcriptionally repressed in treated EMT6 cells with wild type p53 can be identified, followed by RNA-Seq sequencing of cytoplasmic RNA from drug treated or untreated 4T1 and EMT6 cells with or without NMD inhibition to identify CiPAS that are induced specifically in 4T1 cells.

Identifying CiPAS in cells derived from human primary tumors. Freshly obtained triple negative breast cancer tissues from can be used to establish cell lines or primary cultures. These can be treated with chemotherapeutic drugs as described herein to verify the presence of CiPAS identified previously. Also, fresh tissues from biopsies taken from cancer patients 12-24 hours after treatment with chemotherapy can be tested. RNA from these tissues can be analyzed for the CiPAS identified previously to be NMD-resistant to, for example the CiPAS which are the products of retention of the last intron. Also, if the premature translation termination codon generated by abnormal splicing is located in the vicinity of start codons the resulting CiPAS may be resistant to NMD. RNA from biopsies taken from cancer patients treated with 5-FU can be analyzed for the presence of CiPAS which are normally degraded by NMD since due to a partial impairment of translation by 5-FU the NMD in such samples is partially inhibited.

In an aspect, the disclosure provides for testing to establish that CiPAS generate cancer-specific chemo-neoepitopes that elicit immune protection from tumor growth of triple negative breast cancers. This will allow identification of chemo-neoepitopes generated from

CiPAS in chemotherapy-treated mouse breast cancer line 4T1 (p53−/−) but not in EMT6 (p53+/+) by using RNA-Seq, mass-spectrometry and an integrative bioinformatics approach.

The following examples are presented in order to more fully illustrate the preferred embodiments of the invention and should in no way be construed as limiting the scope of the disclosure.

### EXAMPLE 1

Aspects of this Example 1 are demonstrated in Example 2, which demonstrates that treatment of tumor cells with fluorouracil renders tumor cells immunogenic against an antigenically un-related tumor. The following materials and methods are pertinent to the disclosure.

Cells, tissues and chemotherapy treatments. Mouse breast cancer lines (4T1, triple negative, and EMT6, BALB/c), treated as described above, are used. Cells are collected pre- and post-chemotherapy. Approximately 10⁹ cells are used for each measurement. Most of the cells (8×10⁸ cells) can be used for isolation of MHC I-peptide complexes for mass spectrometry, and the remainder for RNA Seq analysis. In addition to cultured cells grown in culture, tumor tissues grown in vivo in absence and presence of the respective chemotherapies can be used. Tumors can be obtained by challenging mice with 200,000 4T1 (in BALB/c mice).

Mass spectrometry of MHC I-associated peptides. H-2-peptide complexes are purified from 10⁸-10⁹ cells/equivalent wet weight of tissues, using immunoaffinity purification. Two independent experiments performed using this method yielded 70-107 known MHC I-bound peptides.

Identification of CiPAS-generated chemo-neoepitopes. Unlike peptides detected in standard tandem-MS proteomic protocols, MHC-associated peptides are not generated by digestion with highly specific enzymes such as trypsin. Instead, they are generated by less well-characterized immunoproteasomes. Furthermore, the peptides of interest do not appear in current proteomics databases since these do not contain aberrant splicing products, particularly if they are targets of NMD. To identify candidate neoepitopes generated by immunoproteasomes from novel CiPAS-encoded polypeptides, a database search approach can be used to build a custom database of candidate neoepitopes generated in silico from open reading frames of putative CiPAS spanning all possible retained introns or alternative reading frames of genes inhibited by p53 activation. The disclosure includes such databases. A recently published meta-analysis of p53 regulated genes identifies more than a thousand genes indirectly downregulated by p53 activation. For the protein translation of each putative CiPAS open reading frame, predictions can be made for cleavage by immunoproteasomes, transporter associated with antigen transport (TAP), ERAP trimming, and MHC-I binding using existing methods such as IEDB, which are trained based on in vitro binding data, and Hidden Markov Models trained from MHC-I eluted peptide sequences recently generated using tandem-MS for human and mouse alleles. A highly stringent set of criteria (e.g. <0.2% peptide FDR) can be employed.

Testing for chemo-neoepitopes for their ability to elicit CD8 and CD4T cells and tumor rejection using p53−/− 4T1 triple-negative breast cancer line or as negative control, p53+/+ EMT6 breast cancer line in syngeneic BALB/c mice can be performed.

Testing for immune response elicited in situ by chemo-neoepitopes (without active immunization). It is expected that CiPAS-elicited chemo-neoepitopes contribute to the immune-stimulatory activity of selected chemotherapies. For additional description, see Example 2 and FIG. 4. Mice bearing 5 day old tumors of each type will not, or will be treated with respective chemotherapies (Carboplatin, 5FU, Cisplatin, Oxaliplatin) at appropriate doses for a single cycle. None of these chemotherapies leads to a complete response in these mouse tumor models. The growth kinetics of tumors are monitored. Considering that (i) chemo-neoepitopes are expected to be generated as the treatment cycle is happening, (ii) T cell responses to chemo-neoepitopes can be reasonably expected to be elicited 7 days after that, and expand afterwards, tumors will be harvested about 14 days after completion of the cycle of treatment. The tumors from un-treated and chemotherapy-treated animals are analyzed as follows, which provides a non-limiting and illustrative protocol: A piece of the tumor is formalin-fixed and analyzed by immunohistochemistry for the number and types of TILS (CD8, CD4 FoxP3−, CD4 FoxP3+, B cells), macrophage and MDSCs. Multiple replicate samples will be analyzed for statistical significance by student's t test with significance of P<0.05. A tumor piece will be rendered into a single cell suspension, from which CD45+ cells (of hematopoietic lineage) are isolated by magnetic separation, and stained with antibodies against CD8, CD45, CD4, FoxP3 and other markers, as well as viability dye. The number of positive cells and their % within the total CD8 cells will be compared between the samples/groups using unpaired Student's t-test. The chemo-neoepitopes generated as described above can include Kd, Dd and Ld-restricted neoepitopes for 4T1. Phycoerythrin-conjugated MHC I-peptide tetramers are prepared for as many of these chemo-neoepitopes as possible. The CD8+ cells, gated in the CD45+ populations, are examined for tetramer-positivity by flow cytometry for each chemo-neoepitope. The CD45+ cells are analyzed by single cell 10X sequencing to assess changes in transcriptional programs of any of the hematopoietic cells post chemotherapy. Briefly, 7-8,000 cells and gel beads (containing reverse transcription and barcoding reagents) are captured in an oil droplet emulsion, in which RNA is reverse transcribed into cDNA with each individual RNA molecule being uniquely tagged. The combined cDNA is amplified for bulk sequencing at a depth of 400 million reads (˜50,000 reads per cell). Through the analyses described above, chemo-neoepitopes ca be defined. The TILs from each tumor, without and with exposure to chemotherapy, are analyzed for stimulation in vitro by the respective chemo-neoepitopes. The CD8 and CD4 responses are assessed by ELISPOT and tetramer binding. Testing for immune response elicited systemically by chemo-neoepitopes (after active immunization). Tumor-bearing mice (5-day post tumor-implantation, although this time window may be modified) as in the previous section, can be immunized twice at a weekly interval with long 17-21 mer extended peptides (pulsed onto bone marrow-derived DCs as adjuvants) containing the individual chemo-neoepitopes identified as described above. Starting one week after the last immunization, mice are treated with a single cycle of the chemotherapy. In addition to the experimental group of mice immunized with chemo-neoepitopes and treated with chemotherapy, the two control groups can include saline-alone injected mice and mice immunized without chemotherapy. An additional control, i.e. mice treated with chemotherapy but not immunized with chemo-neoepitopes, may already have been tested as described above.

The following parameters are measured (at the times indicated below) throughout the period starting from the time the mice are challenged with the tumor, until they are sacrificed.

Kinetics of tumor growth can be monitored every 2-3 d and assessed for significance by student's t test. Using flow cytometry, MEW I-peptide or MEW II-peptide tetramers for chemo-neoepitopes are used to detect antigen-specific CD8+ or CD4+ cells from (250 μl) in blood drawn once weekly. The composition of TILs (CD8, CD4 FoxP3−, CD4 FoxP3+, B cells) in the un-treated and treated mice are tested as described above, on mice on days 10, 20 and 27 and 34 post-challenge. A rationale for the choice of these time points is as follows: day 10 should provide a baseline TIL response in un-treated and treated mice, while day 20 marks the end of the two immunizations, and TILs may or may not be seen in the tumors because the tumors have not yet been treated with chemotherapy and hence, do not express the chemo-neoepitopes. On days 27 and 34, the fullest activity in TILs can be expected. The expectation is that the strongest and statistically significant tumor rejection, CD8 response and TIL infiltration in mice which were immunized and were administered chemotherapy. In another aspect, the disclosure provides a method to identify the CiPAS generated in platinum-based chemotherapy-treated breast cancers, and to test that they naturally elicit CD4/CD8 responses, as follows.

Identify CiPAS in cancer tissues from platinum-based chemotherapy-treated breast cancer patients who have received neoadjuvant chemotherapy, and characterize them for expression of cell cycle genes including CDC25A, CCNA2, CHEK1, NEK2, CDC20, and CDC6. Primary cultures are tested before/after chemotherapy in vitro. These samples are received and analyzed on a rolling basis. These samples can be used as follows: Freeze (not cryopreserve) a portion of samples and have them analyzed for molecular characterization (as described further below), and prepare single cell suspensions of the remainder and cryopreserve them for further analysis. Characterize all samples with respect to HLA (e.g., through a clinical lab and RNA Seq), and p53 status. Identify CiPAS in tumors post chemotherapy and predict chemo-neoepitopes as described above. CiPAS should not be seen in pre-chemotherapy samples, nor in p53 “normal” samples. Characterize samples for expression of cell cycle-related genes CDC25A, CCNA2, CHEK1, NEK2, CDC20, CDC6. These genes are expected to be down-regulated in samples during chemotherapy, but not pre-chemotherapy, and only in p53-deficient samples. Generate primary cultures from cryopreserved single cells, and treat them with chemotherapy in vitro, as described above. Pre- and post-chemotherapy cultures are tested for down-regulation of cell cycle genes as well as for CiPAS as described above. CiPAS-encoded chemo-neoepitopes can be predicted based on individual patients' HLA alleles and examination of blood and/or tumor-infiltrating lymphocytes for CD4 and CD8 responses to them. CiPAS-encoded chemo-neoepitopes can be predicted based on individual patients' HLA alleles and examine blood and/or tumor-infiltrating lymphocytes for CD4 and CD8 responses to them.

In embodiments, analysis descried herein can be carried out with post-resection tumor samples of significant size.

### EXAMPLE 2

This Example demonstrates aspects of the foregoing description, and demonstrates that treatment of tumor cells with fluorouracil, also known as FU or SFU, renders tumor cells immunogenic against an antigenically un-related tumor.

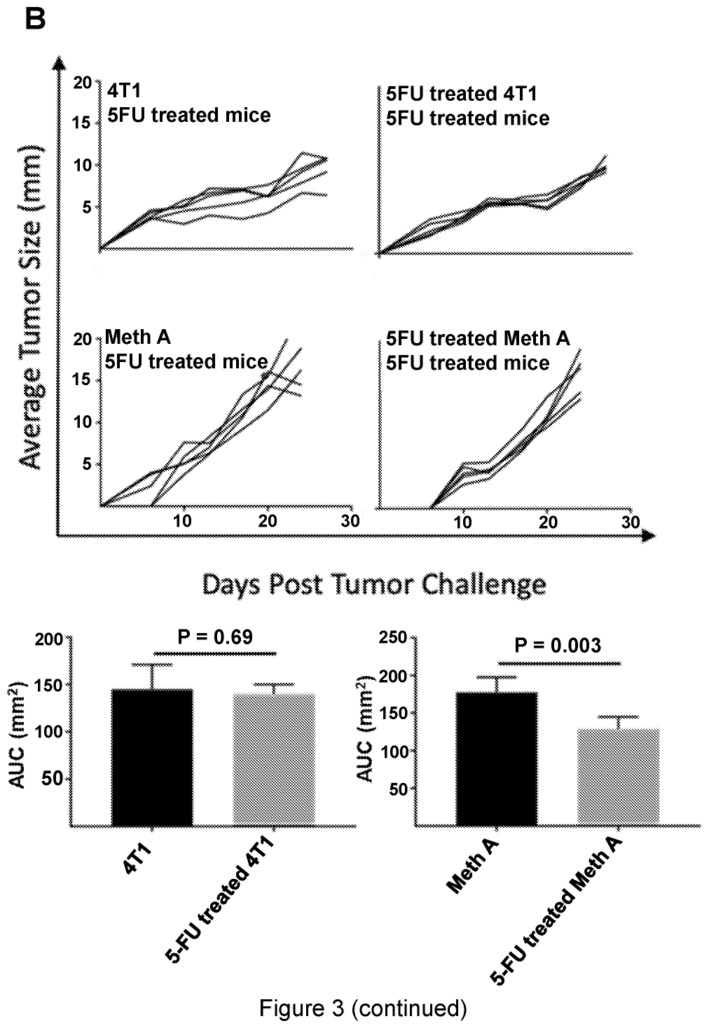

The demonstration is performed using 4T1 and Meth A cancer cells and tumors formed of said cancer cells. As known in the art, 4T1 cells are a breast cancer cell line derived from the mammary gland tissue of mouse. Meth A cancer cells are methyl-cholantrene-induced mouse sarcoma cells. Thus, 4T1 and Meth A cells are non-cross-reactive, i.e. one does not elicit immunity against the other (data not shown). This Example analyzes whether treatment with 5-FU generates new antigenic entities (such as CiPAS), and if treatment of both cell types produces the same antigens in both cells. If this process occurs, the antigens should be processed and presented identically in both cells of the BALB/c haplotype. Accordingly, 5-FU-treated cells of one kind should be able to immunize against 5-FU-treated cells of the other kind. Stated differently, 5-FU treatment should render these non-cross-reactive tumors partially cross-reactive. This effect is demonstrated by the results presented in FIG. 3. In particular, FIG. 3 provides results testing whether immunization of mice with 5-FU-treated 4T1 cells makes the mice at least partially resistant to challenge with 5FU-treated Meth A cells.

To perform this demonstration, the cancer cells were treated (or not treated, as controls) with 5-FU in vitro, but the mice were also administered 5-FU (or not administered, as controls) so as to maintain plasma 5-FU levels in vivo and maintain induction of CiPAS in tumor cells in vivo as well.

First, we tested if treatment of cells in vitro with 5-FU, in and of itself, makes the cancer cells grow differently because of cell death, or another phenomenon. FIG. 3A shows that un-treated and 5-FU treated Meth A cells grow identically (P=0.07). We next tested if un-treated and 5-FU treated 4T1 A cells grow similarly in mice that had themselves not been administered 5-FU or if they had been so treated. FIG. 3B (top panel) shows that un-treated and 5-FU treated 4T1 A cells grow essentially identically in mice that had not been or had been administered 5-FU (P=0.07). Both these serve as controls for the experimental group which is shown in FIG. 3B (bottom panel). In this demonstration, the same mice as shown in FIG. 3B (top panel), were challenged on the other flank with un-treated or 5FU treated Meth A cells. If 5-FU-treated 4T1 cells were eliciting CiPAS, the immunity would develop during one week, and when the same mice were challenged with 5-FU-treated Meth A cells, the latter's growth should be inhibited, since they would also be expressing the same CiPAS as the 5-FU-treated 4T1 cells. This is what was observed in FIG. 3B (bottom panel). 5-FU-treated Meth A cells grow more slowly than un-treated Meth A cells in mice that had previously been immunized with 5-FU-treated 4T1 cells (P=0.003). This effect is not seen in mice that had been previously challenged with 5-FU-untreated 4T1 cells. Thus, this Example and the results presented in FIG. 3 demonstrates the existence of CiPAS and their ability to influence tumor immunity, even in tumors that are distinct from the tumors that express the CiPas.

### EXAMPLE 3

This Example provides non-limiting examples of amino acid sequences identified using the methods described herein.

In one embodiment, a polypeptide comprising neo-antigens comprises a segment of the following amino acid sequence. It was identified as being encoded by a retained intron 6 in mRNA transcribed from the human CCNE2 gene in cells treated with 5FU. The sequence encoded by the retained intron is shown in bold and representative CiPAS segments shown in bold and italics.

(SEQ ID NO: 1)
WGCSKEVWLNMLKKESRYVHDKHFEVLHSDLEPQMRSILLDWLLEVCYSNT
N [symbol] [symbol] NVLSMYSQEFIVHPQEAVFFEVLTVVDCTQSGK

In another embodiment, the following sequence is produced by transcription of the human MCM10 gene, with the 74 novel amino acids shown in black that are encoded by retained intron 12 followed by out of frame sequence of exon 13 until the first nonsense codon.

(SEQ ID NO: 2)
DCEYCQYHVQAQYKKLSAKRADLQSTFSGGRIPKKFARRGTSLKERLCQDG
FYYGGVSSASYAASIAAAVAPKKKIQTTLSNLVVKGTNLIIQETRQKLGIP
QKSLSCSEEFKELMDLPTCGARNLKQHLAKATASGTISCMATRVSLCSLSL
PGHPLSLGFCFRDYGEPKTSHQVHLGLSTLEATEAADVGDEEKEIRRNTEA
ISAELK

The following Table 1 provides predicted-CiPAS and includes polypeptide sequences encoded by introns detected with high confidence in RNA-Seq generated from MDA-MB-231 and MCF10A p53−/− KO cells treated with 5-FU and caffeine and not detected in cells treated with caffeine alone. 38 and 76 CiPAS were detected in MDA-MB-231 and MCF10A p53−/− KO cells, respectively, including 18 shared CiPAS. Certain representative and non-limiting chemo-neoepitopes encoded by the CiPaS sequences are shown in bold, with overlapping amino acids in italics.

TABLE 1

| | Transcript | Retained | Polypeptide sequence encoded by | SEQ ID |
|---|---|---|---|---|
| Cell Line | ID | Intron# | retained intron | NO: |
| Shared | ENST0000 | intron3 | ICVTCKEVKQPQELGRGGLGAGGGRHHLGRRHLSALT | 3 |
| | 0315764 | | HSIPSVKT | |
| Shared | ENST0000 | intron2 | TRLNRRSA | 4 |
| | 0376358 | | | |
| Shared | ENST0000 | intron9 | FRHQMALGD | 5 |
| | 0381019 | | | |
| Shared | ENST0000 | intron1 | MVNSNRKWEARRGPLCVSFSVSPTLVDLFTSSVTPCLSL | 6 |
| | 0396894 | | PHSAFSHIWQLLPTSAACS [symbol] PHPLS [symbol] | |
| | | | SLLSSLCNCPIPLHCSFIFSQPAIQHTFTEHLLCWENIHE | |
| Shared | ENST0000 | intron4 | NHTVAFLGTSDGRILKVWPRLGRGGSGGPVCAQGPYPH | 7 |
| | 0411680 | | RPALAHTGVPHPRWHLLRVRLYPCGDKQESQARPGTVW | |
| | | | RPGQPVRHDPGQGEPDAAPESTLGQVCPVIASQALRTGL | |
| | | | GARGGWGAAELGPSPWSG | |
| Shared | ENST0000 | intron4 | RGCRLTELG [symbol] AGPGVSAGWATGAL | 8 |
| | 0421419 | | | |
| Shared | ENST0000 | intron2 | LEFLTATGVQETFVFCCWKAAQIKEHLL | 9 |
| | 0432982 | | | |
| Shared | ENST0000 | intron2 | GGAAQRSGERRARGRREVEPFGGRSPV [symbol] PAAAA | 10 |
| | 0527430 | | GGGALKRACLGIFSLSLEPVLWTLRSRVLLYHLTFFSFQ | |
| | | | GQGPHLLLSL | |
| Shared | ENST0000 | intron2 | ICAYCHTR | 11 |
| | 0534824 | | | |
| Shared | ENST0000 | intron5 | KQQIESEVSQGSRQTQTPPAWATCPLQREFGQWL | 12 |
| | 0538410 | | | |
| Shared | ENST0000 | intron1 | PYMTPVQVSVPWRGGVRLGEGQAPAASDPQEVIGQI | 13 |
| | 0544738 | | | |
| Shared | ENST0000 | intron1 | ASYPSDHW | 14 |
| | 0563578 | | | |
| Shared | ENST0000 | intron2 | LFVPNLIGECCPRPRAEREGEGPWRAP | 15 |
| | 0564296 | | | |
| Shared | ENST0000 | intron4 | NLPTSLEGLSNLAGQAAPGAPLSSGPLWALLLPWVEGV | 16 |
| | 0577485 | | EPCSSDQYPEGKGWSYVTVTFAIMMMP | |
| Shared | ENST0000 | intron3 | LQGERISGNLDAPEGGFDAILQTAVCTVGTGKGAASLG | 17 |
| | 0582629 | | QG | |
| Shared | ENST0000 | intron1 | SEKHLISGEPLWGAPVLGETAQMGVWRQEDENFHWG | 18 |
| | 0594298 | | GTLCRKRALAERIDLGWDCNCRSERIP | |
| Shared | ENST0000 | intron6 | VRGLVLPGEAWAPCAGMGARRVSPAPSDLDFGFLSFLCI | 19 |
| | 0595185 | | LNPFSLGLAIPPALSWGWLSPRPPPVKAASPPASRRTLRC | |
| | | | HSLSSPPAASAPRPPAVPGMDISGKGHASGDLLEPWPLGR | |
| | | | NLVACWSLWDDGSPMGHWEGGTLGAMEAHVLCDGCC | |
| | | | ELSYPSWCLMGPWVV | |
| Shared | ENST0000 | intron5 | QSFLLLLLLPAcWQCPRTPYAASRDFDVKYVVPSFSAGG | 20 |
| | 0612032 | | LVQAMVTYEGDRNESAVFVAIRNRLHVLGPDLKSVQSL | |
| | | | ATGPAGDPGCQTCAACGPGPHGPPGDTDTKVLVLDPALP | |
| | | | ALVSCGSSLQGRCFLHDLEPQGTAVHLAAPACLFSAHHN | |
| | | | RPDDCPDCVASPLGTRVTVVEQGQASYFYVASSLDAAV | |
| | | | AASFSPRSVSIRRLKADASGFAPGFVALSVLPKHLVSYSIE | |
| | | | YVHSFHTGAFVYFLTVQPASVTDDPSALHTRLARLSATE | |
| | | | PELGDYRELVLDCRFAPKRRRRGAPEGGQPYPVLRVAHS | |
| | | | APVGAQLAIELSIAEGQEVLFGVFVTGKDGGPGVGPNSV | |
| | | | VCAFPIDLLDTLIDEGVERCCESPVHPGLRRGLDFFQSPSF | |
| | | | CPNPVS | |
| MCF10A_ko | ENST0000 | intron2 | LLAAYFFR | 21 |
| | 0254667 | | | |
| MCF10A_ko | ENST0000 | intron1 | EIRGKPIK | 22 |
| | 0319018 | | | |
| MCF10A_ko | ENST0000 | intron1 | DVLEFNQVILPSACPGAQDLREVTGPQGLCGPDCHSFPQ | 23 |
| | 0324340 | | VIIFVKSVQRCMALAQLLVEQNFPAIAIHRGMAQEER | |
| MCF10A_ko | ENST0000 | intron3 | RRARAKAGEGLPGAGAACWGAGQQQGPTSPLPLCCAR | 24 |
| | 0332324 | | SLCREEGQQCCCPAHRELPGQSDLPR | |
| MCF10A_ko | ENST0000 | intron6 | DEEAMEKVSVLFLGSREGEGQKPGSEEQAQKIWGGGTL | 25 |
| | 0342159 | | L | |
| MCF10A_ko | ENST0000 | intron4 | LQELHAKVESQSPLPTWATPLSLWGWGSSLHFWKEYSN | 26 |
| | 0343358 | | HWPVFSGAVTGILTSVA | |
| MCF10A_ko | ENST0000 | intron1 | LIQGLKEVRVLGSRLVIVE | 27 |
| | 0353159 | | | |
| MCF10A_ko | ENST0000 | intron3 | KPTFIKGVSSSPCQVVLKKCLLAGCSGSHL | 28 |
| | 0378600 | | | |
| MCF10A_ko | ENST0000 | intron3 | FSPAPKLGESVRVQESTPHKGKDW | 29 |
| | 0400809 | | | |
| MCF10A_ko | ENST0000 | intron3 | ELRQRKRGREPVALP | 30 |
| | 0404574 | | | |
| MCF10A_ko | ENST0000 | intron2 | PEDYGEEVKATLSFPQSCSFWSRLFEVSCAETRDGWAP | 31 |
| | 0424122 | | DGALEALGSCSLRPCP | |
| MCF10A_ko | ENST0000 | intron2 | VIFILMKVGSSRVHC | 32 |
| | 0430397 | | | |
| MCF10A_ko | ENST0000 | intron1 | DEGLEETGMTPLSGVLKGILRGCHCWTGEGSVGDQ | 33 |
| | 0439557 | | | |
| MCF10A_ko | ENST0000 | intron8 | VSADSMQVVVQCGWPGRLGTPEAAWTPCGKGLKG | 34 |
| | 0441669 | | | |
| MCF10A_ko | ENST0000 | intron2 | TTLDDPLGKGPILFPWDRWNRTLWC | 35 |
| | 0443433 | | | |
| MCF10A_ko | ENST0000 | intron15 | QLRKNQQVRQAVLLSPRWLSHSQVLAPRL | 36 |
| | 0444495 | | | |
| MCF10A_ko | ENST0000 | intron1 | SHLSLAQGESDTPGVGLVGDPGPSRAMPSGLSPGALDSD | 37 |
| | 0450733 | | PVGLGDPLSEISKLLEAGKEGWAREVVVEGNGDAWRDEC | |
| | | | QDFGGL | |
| MCF10A_ko | ENST0000 | intron1 | SPVSKLQVKLEPGTCSSGPAPTTGDVTPQGLLACPSCVL | 38 |
| | 0451363 | | AVASLSCTVPGPLPRPSWEPLARARPPQAPTATAASLALS | |
| | | | SSHSGV | |
| MCF10A_ko | ENST0000 | intron2 | HMVGDKPGGPHPRPGS | 39 |
| | 0455102 | | | |
| MCF10A_ko | ENST0000 | intron1 | AVPAGAKM | 40 |
| | 0466179 | | | |
| MCF10A_ko | ENST0000 | intron4 | VLPSFYQVRRFLCAVSWQERESSPHACPPIPLGVLLRGLR | 1 |
| | 0467949 | | WHF | |
| MCF10A_ko | ENST0000 | intron1 | GILAALQGA | 42 |
| | 0471530 | | | |
| MCF10A_ko | ENST0000 | intron1 | QELRALQGRLRAQGLGPALLHRPLFAFPDAVRAPSGS | 43 |
| | 0472410 | | | |
| MCF10A_ko | ENST0000 | intron1 | PAIAFGGRWAPPPASAPPPSPAPTQRPQPFSPLPSDSHRCL | 44 |
| | 0473532 | | FVRPQAVVPGLVLPPPGSPPTCPPSHLLSLDVRPH | |
| MCF10A_ko | ENST0000 | intron4 | AAGGSSEGKGMAGG | 45 |
| | 0488731 | | | |
| MCF10A_ko | ENST0000 | intron2 | DMAIAMAVSYTPAWGLQGTQELTMVGCAEGQG | 46 |
| | 0489263 | | | |
| MCF10A_ko | ENST0000 | intron2 | PRNYLNTLSTALNILEKYGRNLLSPQRPRYWRGVKFNNP | 47 |
| | 0491351 | | VFRSTVDAVQVNPLALWERGLGFG | |
| MCF10A_ko | ENST0000 | intron3 | HQRFQFSRWVPIWRRPAVSLK | 48 |
| | 0507555 | | | |
| MCF10A_ko | ENST0000 | intron9 | QTLGSLKACLPVLTPSIHQV | 49 |
| | 0510698 | | | |
| MCF10A_ko | ENST0000 | intron27 | AQVPQASGEQPRGNGANPPGAPPEVEPSSGNPSPQQAAS | 50 |
| | 0514566 | | VLLPRCRLNPDSSWAPKRVATASPFSGLQKAQSVHSLVP | |
| | | | QGEKPDGFSARVQGVVVNTCTCWRREGLVVVP | |
| MCF10A_ko | ENST0000 | intron6 | FSQLGFIGVPLPLPDGAFQSHLPLPSSLPVPLCPL | 51 |
| | 0518980 | | | |
| MCF10A_ko | ENST0000 | intron1 | PTIGYCQVRGLTGAQRQHLPPEVHRSRSLWSQACVVTLP | 52 |
| | 0522472 | | CVPRKEPSRKRCNLKTVLIAHRIPMPNRSRRLCTFV | |
| MCF10A_ko | ENST0000 | intron2 | MGHRYVEGLIYIALVTVNKALNNRGLTPSEF | 53 |
| | 0523137 | | | |
| MCF10A_ko | ENST0000 | intron1 | EVCRVCQVSAAPGSTA | 54 |
| | 0524749 | | | |
| MCF10A_ko | ENST0000 | intron13 | PEVLQKGVAYDSSADWFSLGCMLFKLLRG | 55 |
| | 0526285 | | | |
| MCF10A_ko | ENST0000 | intron1 | TGSGFRVGEGRGHGWGQGKDRHQARWES | 56 |
| | 0526825 | | | |
| MCF10A_ko | ENST0000 | intron7 | CCKTLDQVSVVLSTSNTKVVKHSIRSVIEAKVR | 57 |
| | 0527971 | | | |
| MCF10A_ko | ENST0000 | intron1 | FEEPELRVGPRSRPRPGPAQPPSLPLQLGSVAPSVSLGVSY | 58 |
| | 0531042 | | AGPG | |
| MCF10A_ko | ENST0000 | intron4 | LWEELGHR | 59 |
| | 0531839 | | | |
| MCF10A_ko | ENST0000 | intron5 | AVMRILSGERGCQG | 60 |
| | 0532846 | | | |
| MCF10A_ko | ENST0000 | intron2 | ETDFGGDVSLGTPGPEGGGAGGLDSWV | 61 |
| | 0538643 | | | |
| MCF10A_ko | ENST0000 | intron3 | KNYRSRAVSGAGWRPRSESWLCGAQRQMACGLGSHSD | 62 |
| | 0546898 | | NGGARTQIALLTPSRTPAGIFI | |
| MCF10A_ko | ENST0000 | intron4 | TRLCGDNK | 63 |
| | 0554477 | | | |
| MCF10A_ko | ENST0000 | intron1 | SLKQIFQVRPRSGLGVSV | 64 |
| | 0561922 | | | |
| MCF10A_ko | ENST0000 | intron3 | ENAPALAVLPIYSQLPSDLQAKIFQKVCQQECPGVALGV | 65 |
| | 0562774 | | L | |
| MCF10A_ko | ENST0000 | intron3 | GSGSSYSGSSSRSRSSPSPCASVSPSPSGPGSPRSSQCGLGC | 66 |
| | 0564341 | | LPQVGWGPSGSESPWGCGRLRLKL | |
| MCF10A_ko | ENST0000 | intron1 | LLLPRVRGELTELERATAGAGSGSGRGRARGRGEAGLG | 67 |
| | 0567434 | | VAERAGWGGGGEPGLGEGGGSAYLSFSIRHRSAPTRHTH | |
| | | | KSAKPRRPPRSLIERCSGEDSVSAPPSGLPLPQFLQRPDPF | |
| | | | SSRCPPRRGLLSPAASFL | |
| MCF10A_ko | ENST0000 | intron2 | LAAVLKEVCDA | 68 |
| | 0571587 | | | |
| MCF10A_ko | ENST0000 | intron2 | TVLQAGKALVSGGGGGGGGRGGLGCPHVVCLPEAWLT | 69 |
| | 0572759 | | AGPLSLRVERTVRTRRSWGMRP | |
| MCF10A_ko | ENST0000 | intron2 | FTAQQRQAWDR | 70 |
| | 0573723 | | | |
| MCF10A_ko | ENST0000 | intron1 | KGTSLDPR | 71 |
| | 0578558 | | | |
| MCF10A_ko | ENST0000 | intron1 | SSQDEVGARNHPDWVSMGGVVA | 72 |
| | 0588359 | | | |
| MCF10A_ko | ENST0000 | intron5 | AAAAAVGVRAPPPIRPVAQAGPGAPVCPSPLIPWAWRW | 73 |
| | 0588513 | | TQPSSRGQERGRCGRPWFL | |
| MCF10A_ko | ENST0000 | intron5 | MVSSGSREGFGGGGRSVGNSGVCRPTQGSVVSHNRSLW | 74 |
| | 0591474 | | TERRESMGRMMNHLGVLW | |
| MCF10A_ko | ENST0000 | intron3 | LIPQQLLVRPAPPTPSTLSTPMPG | 75 |
| | 0600793 | | | |
| MCF10A_ko | ENST0000 | intron2 | GSKHLHYWSAALPSGRDWEKR | 76 |
| | 0606394 | | | |
| MCF10A_ko | ENST0000 | intron1 | FDDFSRDLCVQALLDIMDMFCDRLR | 77 |
| | 0614384 | | | |
| MCF10A_ko | ENST0000 | intron3 | AKGIMQLVGQAEPESVLLVHGEAKKMEFLKQKIEQELRR | 78 |
| | 0618806 | | QPGGRRGAHLGSARAAWAEALSSPGVQGSTATCRPMAR | |
| | | | R | |
| MDA-MB-231 | ENST0000 | intron4 | FSGEAYEVGGVVVLNVEEGVPDGLGIS | 79 |
| | 0262396 | | | |
| MDA-MB-231 | ENST0000 | intron10 | KEEVFEKVGVPETVLGAGLGKSASAP | 80 |
| | 0297979 | | | |
| MDA-MB-231 | ENST0000 | intron2 | YAGGAAQVSHRSLGKGVVVERRSASAPAIGRDLPLGARY | 81 |
| | 0350303 | | LPTAQSPKVGERASQGALGRN | |
| MDA-MB-231 | ENST0000 | intron9 | APDPAHYR | 82 |
| | 0374491 | | | |
| MDA-MB-231 | ENST0000 | intron1 | RGESIYWGPTADSQD [symbol] GEEFPPYPTRGPRPTSRRPPPP | 83 |
| | 0378818 | | VASLPSRP | |
| MDA-MB-231 | ENST0000 | intron8 | AFTILAKVSVRSGPGPAGGPGCILSPCSTVSSG | 84 |
| | 0393994 | | | |
| MDA-MB-231 | ENST0000 | intron8 | QQDRELKVNMQTGGAGGMELVRGRTRGLCLLHIEPAGG | 85 |
| | 0406172 | | GPGQPSWVQESFVLGVEDLYPLHPGLEGKGHHVFAWL | |
| | | | GWVMVVAPCGAWQVVVSTCPDPEVQQWARPPTPTLFPW | |
| | | | VTRGRGCPSGTPWLPDF | |
| MDA-MB-231 | ENST0000 | intron5 | RRFIPPARCLLLLSPHSLPKASRILIKSRNRDLEPGDRDAS | 86 |
| | 0438274 | | SPSLLALSEKMYSLIPGPSPSSMDFFL | |
| MDA-MB-231 | ENST0000 | intron1 | GPSEPTER | 87 |
| | 0450095 | | | |
| MDA-MB-231 | ENST0000 | intron4 | LWNQAKEVNK | 88 |
| | 0455748 | | | |
| MDA-MB-231 | ENST0000 | intron1 | LVMIIVRGAS | 89 |
| | 0487146 | | | |
| MDA-MB-231 | ENST0000 | intron2 | RCARTDKVGGGLLCPSPAAMSSTWPTFQLSVLACYAVSE | 90 |
| | 0535067 | | HIMMYKSN | |
| MDA-MB-231 | ENST0000 | intron1 | WQYGPCTGENPPSPTEAPLPASHPHVSEVTQALA | 91 |
| | 0539074 | | | |
| MDA-MB-231 | ENST0000 | intron4 | ASAADPQVRPLTLPSELPTVRHRSSPSIRPLRSAQHLPART | 92 |
| | 0558046 | | PTCLLPRPRSASLRPC | |
| MDA-MB-231 | ENST0000 | intron1 | YSAPDLLG | 93 |
| | 0561668 | | | |
| MDA-MB-231 | ENST0000 | intron3 | EFRKHGIGKGSRARGLAPTPPPCPTPPPSHLHPVSSLAPTT | 94 |
| | 0570819 | | LAREHPSVSSSILVPVLGQTFIIPPLPAHPPPSAILFPHAPRP | |
| | | | FCCSLAQPPGWS | |
| MDA-MB-231 | ENST0000 | intron1 | SDPDSDKGRSAGRGHIGGRPFLGASYEP | 95 |
| | 0585656 | | | |
| MDA-MB-231 | ENST0000 | intron1 | AQLMNCHVSAGTRHKVLLRRLLASFFDR | 96 |
| | 0585663 | | | |
| MDA-MB-231 | ENST0000 | intron1 | GRTVPSDVSPNNEYSPAGTPPPVGISSSPPWISPASMNTSH | 97 |
| | 0593686 | | SCISLPPSCIFSLCPLISVSPWEHMPPVSGPPFFFFF | |
| MDA-MB-231 | ENST0000 | intron2 | RINKTSPGELGQRGS | 98 |
| | 0597811 | | | |

1. A method for identifying antigens comprising amino acid sequences for use in a cancer vaccine, the method comprising:
i) exposing cancer cells to a chemotherapeutic agent that damages DNA;
ii) determining open reading frames encoded by mRNA transcribed from a gene in the cancer cells of i);
iii) comparing the open reading frames of the mRNA of i) to open reading frames encoded by mRNA transcribed from the gene in the cancer cells that were not exposed to the chemotherapeutic agent,
determining a different open reading frame encoded by the mRNA of i) and an open reading frame of the mRNA of ii), wherein the different open reading frame encoded by the mRNA of i) encodes a contiguous amino acid sequence comprising the sequence of the antigen for use in the cancer vaccine.

2. The method of claim 1, wherein the open reading frames of the mRNA of i) are encoded by an a sequence from an intron, wherein said sequence is retained in the mRNA after splicing.

3. The method of claim 1, comprising repeating i), ii) and iii) from a plurality of distinct cancer cells to determine a plurality of distinct antigen sequences.

4. The method of claim 1, wherein the cancer cells comprise a mutated p53 protein.

5. The method of claim 1, wherein the cancer cells are human cancer cells.

6. The method of claim 1, further comprising producing a peptide comprising a contiguous amino acid sequence comprising the sequence of the antigen.

7. The method of claim 6, wherein the peptide consists of from 9-11 contiguous amino acids selected from the amino acid sequences elected from the amino acid sequence presented in Table 1.

8. The method of claim 6, further comprising mixing the peptide with a pharmaceutically acceptable agent to obtain a vaccine formulation.

9. A method comprising administering to an individual in need thereof a vaccine comprising a peptide identified and produced according to the method of claim 6.

10. The method of claim 9, further comprising administering to the individual a chemotherapeutic agent that damages DNA such that a polypeptide comprising the sequence of the antigen is produced by cancer cells in the individual.

11. The method of claim 10, wherein the individual has not previously been treated with the chemotherapeutic agent before administering the vaccine.

12. The method of claim 9, wherein the peptide comprises an amino acid sequence comprising from 9-11 contiguous amino acids selected from the amino acid sequences presented in Table 1.

13. A vaccine formulation comprising a peptide identified according to the method of claim 5, the formulation further comprising at least one pharmaceutically acceptable agent.

14. The vaccine formulation of claim 13, further comprising an adjuvant.

15. The vaccine formulation of claim 13, wherein the peptide comprises from 9-11 contiguous amino acids selected from the amino acid sequences selected from the amino acid sequence presented in Table 1.

16. The vaccine formulation of claim 15, wherein the peptide consists of a 9-11 amino acid segment selected from the amino acid sequences presented in Table 1.

17. An expression vector encoding a polypeptide comprising the amino acid sequence of a peptide identified by the method of claim 1.

18. The expression vector of claim 17, wherein the peptide comprises an amino acid sequence selected from the amino acid sequences presented in Table 1.

19. The expression vector of claim 18, wherein the peptide comprises a 9-11 amino acid segment selected from the amino acid sequences presented in Table 1.

20. A library of peptides comprising a plurality of peptides identified by the method of claim 1.
